(19) United States
(12) Patent Application Publication
(10) Pub. No.: US 2025/0017770 A1
(43) Pub. Date: Jan. 16, 2025

(54) DEVICES, SYSTEMS, AND METHODS RELATED TO IMPLANTS FOR THE TREATMENT OF GLAUCOMA

(71) Applicant: Duke University, Durham, NC (US)

(72) Inventors: Rami Gabriel, Durham, NC (US); Pratap Challa, Durham, NC (US)

(21) Appl. No.: 18/771,330

(22) Filed: Jul. 12, 2024

Related U.S. Application Data
(60) Provisional application No. 63/526,303, filed on Jul. 12, 2023.

(51) Int. Cl. A61F 9/00; A61F 9/007

(52) U.S. Cl. CPC A61F 9/0017; A61F 9/00781

(57) ABSTRACT

The present disclosure provides devices, systems, and methods relating to the treatment of glaucoma. In particular, the present disclosure is directed to hydrogel implants for use in glaucoma drainage devices to deliver therapeutic agents to a subject's eye and to obviate the need for tying off a drainage tube, thus enhancing therapeutic outcomes.

### RELATED APPLICATIONS

This application claims priority to and the benefit of U.S. Provisional Patent Application No. 63/526,303 filed Jul. 12, 2023, which is incorporated herein by reference in its entirety for all purposes.

### FIELD

This present disclosure provides devices, systems, and methods relating to the treatment of glaucoma. In particular, the present disclosure is directed to hydrogel implants for use in glaucoma drainage devices to deliver therapeutic agents to a subject's eye and to obviate the need for tying off a drainage tube, thus enhancing therapeutic outcomes.

### BACKGROUND

The efficacy of drug-eluting implants has increased with the use of dissolvable hydrogels. For example, Dextenza is a corticosteroid used to treat ocular inflammation (e.g., occurring after cataract surgery) and is one of the more common anterior segment implants. More specifically, Dextenza is formulated as a dexamethasone-eluting polyethylene glycol (PEG) hydrogel, and it has been shown to be effective in reducing inflammation and improving patient outcomes. The use of Dextenza reduced the need for rescue medication and patients reported better pain control compared to topical steroids. Also, Dextenza has also been shown to reduce inflammation in patients undergoing trabeculectomy surgery. While the usage of an implant in the canaliculus has been well studied, there has only been one report of using Dextenza subconjunctivally. Overall, integrating therapeutic agents such as Dextenza into ocular medical devices has not been fully explored.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Embodiments of the present disclosure include a hydrogel implant for treating glaucoma in a human subject. In accordance with these embodiments, the implants described herein include at least one therapeutic agent and are formulated to occupy a portion of a lumen of a glaucoma drainage device.

In some embodiments, the hydrogel comprises polyethylene glycol (PEG). In some embodiments, the PEG comprises polyethylene glycol diacrylate (PEGDA), polyethylene glycol dimethyl acrylate (PEGDMA), and/or polyethylene glycol divinyl ether (PEGDVE), or any derivatives thereof. In some embodiments, the PEG comprises a molecular weight ranging from 100 to about 20,000. In some embodiments, the PEG comprises from about 40% to about 90% PEG.

In some embodiments, the implant comprises riboflavin-5-phosphate and/or triethanolamine.

In some embodiments, the at least one therapeutic agent included in the implants of the present disclosure is selected from an antibiotic, a small molecule inhibitor, a steroid, and/or a biologic drug. In some embodiments, the antibiotic is selected from the group consisting of Tobramycin, Vancomycin, Ciprofloxacin, Ofloxacin, and Moxifloxacin. In some embodiments, the small molecule inhibitor is selected from the group consisting of Rhopressa, Timolol, Brimonidine, and Dorzolamide. In some embodiments, the biologic drug is an antibody selected from bevacizumab and ranibizumab.

In some embodiments, the implant is formulated to remain stationary in the lumen of the glaucoma drainage device at pressures up to and including 400 mmHg.

In some embodiments, the glaucoma drainage device comprises a plate and a drainage tube, and wherein the implant is formulated to occupy a distal portion of the lumen of the drainage tube.

In some embodiments, the implant is formulated to dissolve and release the at least one therapeutic agent over a period of about 30 days to about 90 days.

In some embodiments, the implant is in contact with a ripcord, wherein pulling the ripcord facilitates removal of the implant from the lumen of the glaucoma drainage device.

Embodiments of the present disclosure also include a glaucoma drainage device for treating glaucoma in a human subject. In accordance with these embodiments, the device includes a hydrogel implant formulated to occupy a portion of a lumen of a glaucoma drainage device and includes at least one therapeutic agent.

In some embodiments, the glaucoma drainage device comprises a plate and a drainage tube, and the implant is formulated to occupy a distal portion of the lumen of the drainage tube.

In some embodiments, the hydrogel comprises polyethylene glycol (PEG). In some embodiments, the PEG comprises polyethylene glycol diacrylate (PEGDA), polyethylene glycol dimethyl acrylate (PEGDMA), and/or polyethylene glycol divinyl ether (PEGDVE), or any derivatives thereof.

In some embodiments, the at least one therapeutic agent is selected from an antibiotic, a small molecule inhibitor, a steroid, and/or a biologic drug.

In some embodiments, the implant is formulated to remain stationary in the lumen of the glaucoma drainage device at pressures up to and including 400 mmHg. In some embodiments, the implant is formulated to dissolve and release the at least one therapeutic agent over a period of about 30 days to about 90 days.

In some embodiments, the implant is in contact with a ripcord, wherein pulling the ripcord facilitates removal of the implant from the lumen of the glaucoma drainage device.

Embodiments of the present disclosure also include a method of generating a glaucoma drainage device, which includes positioning a hydrogel implant into a distal portion of a drainage tube of the glaucoma drainage device. In some embodiments, the implant comprises at least one therapeutic agent.

### BRIEF DESCRIPTION OF THE DRAWINGS

The accompanying figures are provided by way of illustration and not by way of limitation.

FIG. 1: a perspective view of a glaucoma drainage device positioned in a subject's eye that includes a hydrogel implant in the distal portion of the lumen of a drainage tube, according to one embodiment of the present disclosure.

FIG. 2: a topside perspective view of a glaucoma drainage device that includes a hydrogel implant in the distal portion of the lumen of a drainage tube, according to one embodiment of the present disclosure.

FIG. 3: an underside perspective view (in contact with subject's eye) of a glaucoma drainage device that includes a hydrogel implant in the distal portion of the lumen of a drainage tube, according to one embodiment of the present disclosure.

FIG. 4: a perspective view of a glaucoma drainage device that includes a hydrogel implant in the distal portion of the lumen of a drainage tube, as well as a ripcord to facilitate removal of the implant, according to one embodiment of the present disclosure.

FIG. 5: a schematic representation of the materials and process for generating the hydrogel implants, according to one embodiment of the present disclosure.

FIG. 6: representative results of swelling ratios of the various PEGDA hydrogel implants at four different timepoints, according to one embodiment of the present disclosure.

FIGS. 7A-7D: a schematic representation of a testing device designed to simulate intraocular pressures in the subconjunctival space exerted on a glaucoma drainage device, according to one embodiment of the present disclosure (FIG. 7A); representative images of a glaucoma drainage device comprising a hydrogel implant and a ripcord for testing intraocular pressures, according to one embodiment of the present disclosure (FIG. 7B); representative results of pressure testing of the hydrogel implant within the lumen of the drainage device, according to one embodiment of the present disclosure (FIG. 7C); and representative results of pressure testing with a ripcord in place, according to one embodiment of the present disclosure (FIG. 7D).

### DETAILED DESCRIPTION

Section headings as used in this section and the entire disclosure herein are merely for organizational purposes and are not intended to be limiting.

All publications, patent applications, patents and other references mentioned herein are incorporated by reference in their entirety.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

“About” is used to provide flexibility to a numerical range endpoint by providing that a given value may be “slightly above” or “slightly below” the endpoint without affecting the desired result.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise—Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

The terms “biocompatible” and “biocompatibility” as used herein generally refer to a material or a property of a material that does not cause an adverse reaction in a subject when placed in proximity to a portion of the subject's body (e.g., eye or portion thereof) or when in contact with one or more of the subject's tissues (internally or externally positioned). Adverse reactions include inflammation, infection, fibrotic tissue formation, cell death, thrombosis, and the like.

The terms “biodegradable” and “bioabsorbable” as used herein generally refer to a material or a property of a material that is capable of being broken down (catabolized and/or metabolized, absorbed and/or excreted) inside a subject by any means, without causing or being associated with a significant adverse reaction in a subject. For example, a biodegradable material and a device made of biodegradable material does not persist within a subject's body long-term (e.g., eye or portion thereof), but is substantially absorbed and/or broken down by the subject's body in a manner that has no significant detrimental physiological and/or biochemical effects on the subject.

“Implant” and “body implant” generally refer to an article or device that is placed entirely or partially into a subject (e.g., eye or portion thereof), for example by a surgical procedure or medical intervention, for any period of time.

Unless otherwise defined herein, scientific and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Implants Glaucoma Drainage Devices

Embodiments of the present disclosure relate generally to devices, systems, and methods for the treatment of glaucoma. In particular, the present disclosure is directed to hydrogel implants for use in glaucoma drainage devices to deliver therapeutic agents to a subject's eye and to obviate the need for tying off a drainage tube, thus enhancing therapeutic outcomes.

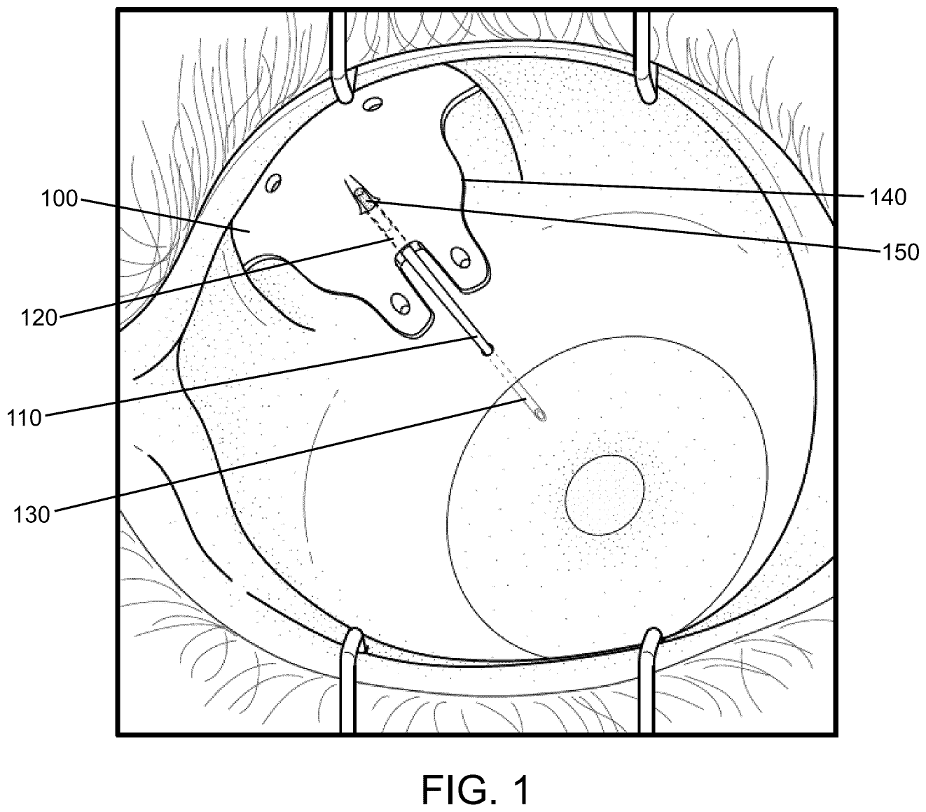

As described further herein, glaucoma drainage devices (GDDs) are traditionally tied off using a dissolvable stitch tied over the tube with an intraluminal stent, called a “rip cord,” placed within the tube lumen. This cord serves both as a way to help tie off the tube easier and as a fail-safe to be pulled out if the pressure rises and is recalcitrant to medical therapy. The remaining ripcord is tunneled into the conjunctiva. Embodiments of the present disclosure explore an alternative to tying off the tube using a dissolvable drug-eluting implant that is placed within the lumen of the tube (FIG. 1). The purpose of using a dissolvable drug-eluting implant within the tube lumen of GDDs is to decrease the drop burden of high-risk patients, reduce risk for exposure complications, as well as helping to localize delivery of a therapeutic agent. Additionally, a rip cord can still be used in conjunction with the intraluminal implants of the present disclosure, keeping the fail-safe mechanism to open the tube as a precaution.

In accordance with these embodiments, the implants of the present disclosure are designed for use with any glaucoma drainage devices (e.g., Ahmed Clearpath 350, or Baerveldt 350). One exemplary embodiment is illustrated in FIGS. 1-4. With reference to FIGS. 1-4, a glaucoma drainage device 100 is positioned in the eye according to established medical practices. The device 100 generally includes a plate 140, as well as a drainage tube 110 for draining fluid that builds up in the eye, which is one hallmark of glaucoma. This buildup of fluid results in an increase in intraocular pressure (IOP) that can damage the optic nerve and lead to vision loss. The drainage tube 110 of the glaucoma drainage device 100 has a proximal end 130 that is the entry point for fluid drainage from the eye, as well as a distal end 120 that is positioned near the plate 140. In some embodiments, the distal end 120 of the drainage tube 110 can be configured to have a modified shape or structure that is expanded as compared to the diameter of the rest of the tube. In this manner, the distal end 120 of the drainage tube 110 is adjustable to accommodate, for example, the type, dosage, or delivery kinetics of the therapeutic agent(s) being delivered to the subject's eye. In some embodiments, the distal end 120 of the drainage tube 110 can be coupled to a reservoir that contains the therapeutic agent(s) in order to deliver an increased amount or concentration of the therapeutic agent(s).

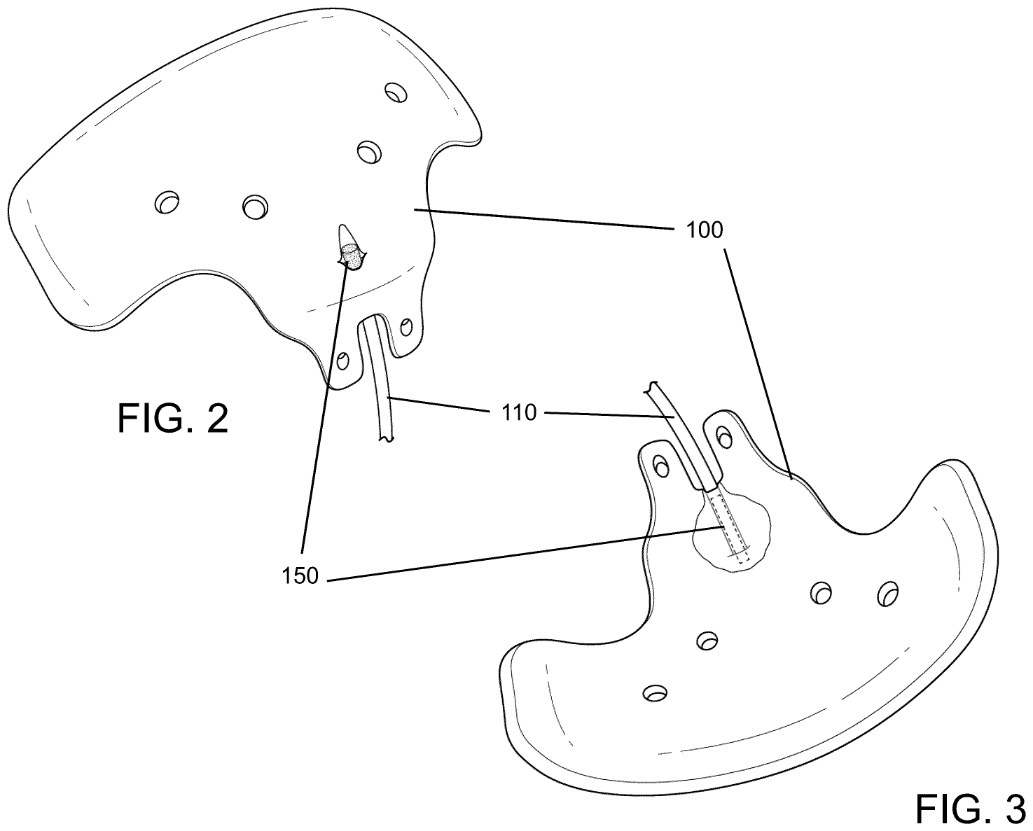

As described further herein, the distal end 140 of the drainage tube 110 can include a hydrogel implant 150. For example, as shown in FIGS. 2 and 3, the hydrogel gel implants 150 of the present disclosure are configured for insertion into the distal end 150 of a drainage tube of a glaucoma drainage device 100. In some embodiments, the hydrogel comprises polyethylene glycol (PEG). In some embodiments, the PEG comprises polyethylene glycol diacrylate (PEGDA), polyethylene glycol dimethyl acrylate (PEGDMA), and/or polyethylene glycol divinyl ether (PEGDVE), or any derivatives thereof. As would be understood by one of ordinary skill in the art based on the present disclosure, various PEG formulations can be used to generate the hydrogel implants of the present disclosure, and the precise hydrogel PEG formulation depends on the type of glaucoma drainage device, the type of therapeutic agent that is formulated in the implant, the specific dissolving rates desired, and the like.

For example, in some embodiments, the PEG comprises a molecular weight ranging from 100 to about 20,000. In some embodiments, the PEG comprises a molecular weight ranging from 500 to about 20,000. In some embodiments, the PEG comprises a molecular weight ranging from 1000 to about 20,000. In some embodiments, the PEG comprises a molecular weight ranging from 2000 to about 20,000. In some embodiments, the PEG comprises a molecular weight ranging from 4000 to about 20,000. In some embodiments, the PEG comprises a molecular weight ranging from 6000 to about 20,000. In some embodiments, the PEG comprises a molecular weight ranging from 8000 to about 20,000. In some embodiments, the PEG comprises a molecular weight ranging from 10,000 to about 20,000. In some embodiments, the PEG comprises a molecular weight ranging from 12,000 to about 20,000. In some embodiments, the PEG comprises a molecular weight ranging from 14,000 to about 20,000. In some embodiments, the PEG comprises a molecular weight ranging from 16,000 to about 20,000. In some embodiments, the PEG comprises a molecular weight ranging from 18,000 to about 20,000. In some embodiments, the PEG comprises a molecular weight ranging from 100 to about 15,000. In some embodiments, the PEG comprises a molecular weight ranging from 100 to about 10,000. In some embodiments, the PEG comprises a molecular weight ranging from 100 to about 5000. In some embodiments, the PEG comprises a molecular weight ranging from 100 to about 2500. In some embodiments, the PEG comprises a molecular weight ranging from 100 to about 1000. In some embodiments, the PEG comprises a molecular weight ranging from 1000 to about 10,000. In some embodiments, the PEG comprises a molecular weight ranging from 5000 to about 15,000. In some embodiments, the PEG comprises a molecular weight ranging from 8000 to about 16,000. In some embodiments, the PEG comprises a molecular weight ranging from 10,000 to about 15,000.

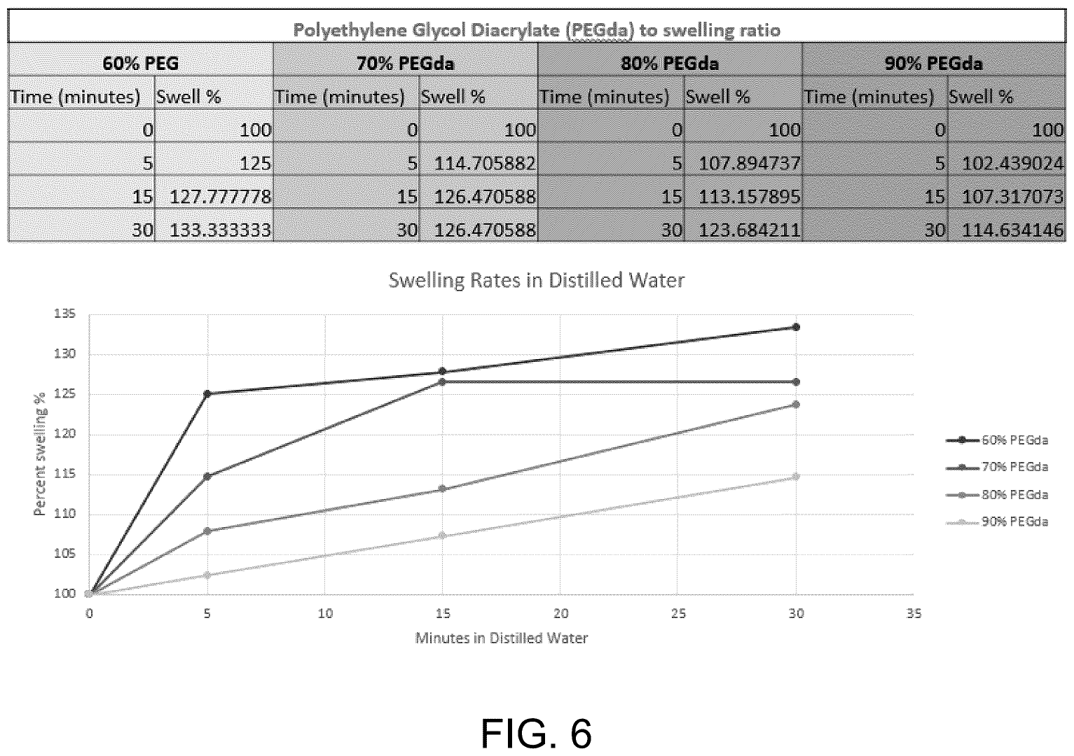

In some embodiments, the PEG comprises from about 40% to about 90% PEG. In some embodiments, the PEG comprises from about 50% to about 90% PEG. In some embodiments, the PEG comprises from about 60% to about 90% PEG. In some embodiments, the PEG comprises from about 70% to about 90% PEG. In some embodiments, the PEG comprises from about 80% to about 90% PEG. In some embodiments, the PEG comprises from about 40% to about 80% PEG. In some embodiments, the PEG comprises from about 40% to about 70% PEG. In some embodiments, the PEG comprises from about 40% to about 60% PEG. In some embodiments, the PEG comprises from about 40% to about 50% PEG. In some embodiments, the PEG comprises from about 50% to about 75% PEG. In some embodiments, the PEG comprises from about 60% to about 70% PEG. In accordance with these embodiments, various percentages of PEG correspond to different swelling ratios, which can inform the choice of a particular hydrogel formulation over another. For example, as shown in FIG. 6, as the percentage of PEG increases (e.g., PEGDA) from 60% to 90%, the percent swelling decreases (over the four timepoints shown). Therefore, PEG percentages can be varied to produce different swelling rates, which can influence the process for generating the hydrogel implants of the present disclosure.

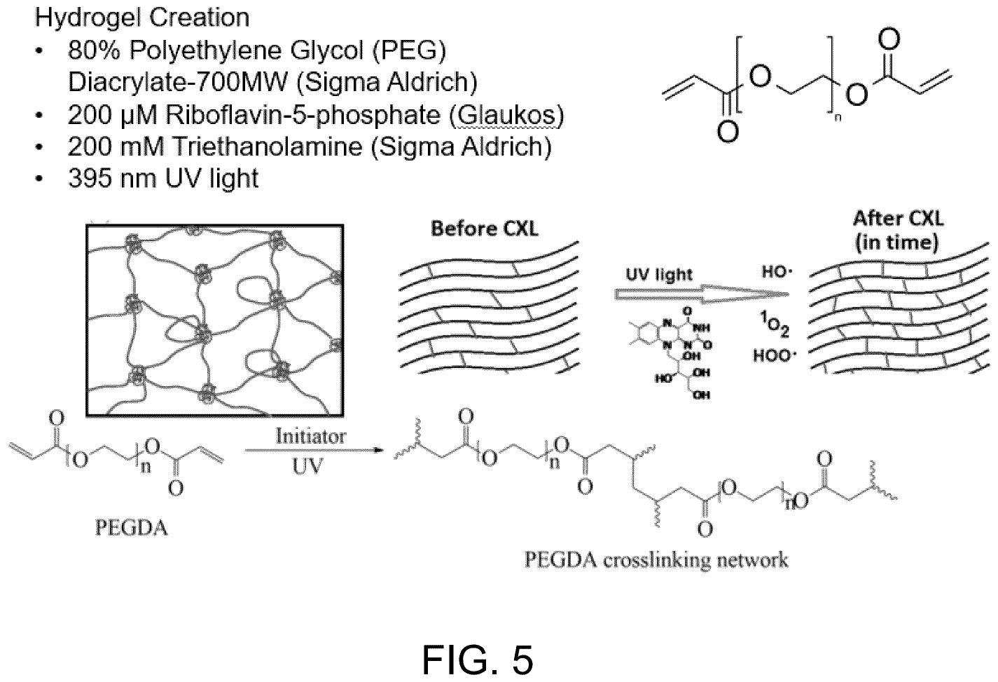

In accordance with these embodiments, the hydrogel implants of the present disclosure can be formulated with a therapeutic agent designed to treat one or more systems in a subject's eye. In some embodiments, the symptom is associated with glaucoma. In some embodiments, the symptom is associated with a disease or disorder that is not characteristic of glaucoma. In other embodiments, the implant can also be formulated with a pharmaceutically acceptable carrier, adjuvant, and/or excipient. For example, in some embodiments, the implant comprises riboflavin-5-phosphate and/or triethanolamine. One exemplary process for generating a hydrogel according to the methods of the present disclosure is shown in FIG. 5.

In some embodiments, the hydrogel implant is formulated to dissolve over a given period of time (e.g., the length of time for a glaucoma drainage device, after being implanted into a subject's eye, to reach a stabilize position). This desired length of time for the therapeutic agent to dissolve will be among the factors affecting the particular formulation of the hydrogel. In some embodiments, the hydrogel implant will dissolve and release the therapeutic agent into a subject's eye, and after the implant dissolves, the drainage tube of the glaucoma drainage device provides an unobstructed path for fluid to drain from the device.

As would be understood by one of ordinary skill in the art based on the present disclosure, the therapeutic agent or agents that can be included in the hydrogel implants of the present disclosure can include any therapeutic agent that has been shown to be safe for administration to a subject's eye. For example, in some embodiments, the therapeutic agent included in the implants of the present disclosure is an antibiotic, a small molecule inhibitor, a steroid, and/or a biologic drug (e.g., antibody). In some embodiments, the antibiotic is selected from the group consisting of Tobramycin, Vancomycin, Ciprofloxacin, Ofloxacin, and Moxifloxacin. In some embodiments, the small molecule inhibitor is selected from the group consisting of Rhopressa, Timolol, Brimonidine, and Dorzolamide. In some embodiments, the biologic drug is an antibody selected from bevacizumab and ranibizumab.

In one exemplary embodiment, the therapeutic agent that can be included in the hydrogel implants of the present disclosure is a steroid, such as dexamethasone. For example, in one embodiment, Dextenza can be used as hydrogel implant. While currently available formulations of Dextenza are configured to be too large to be placed into the lumen of a glaucoma drainage tube, the use of PEG hydrogels is becoming more common and it is possible to create a new implant that releases steroid or intraocular pressure (IOP) lowering medications (e.g., OTX-TIC). PEG-based implants of the present disclosure are formulated to be biocompatible and biodegradable. The rate of breakdown can be controlled by adjusting the size of the PEG molecules and the degree of PEGylation. For example, PEGylated liposomal drug systems for steroid release are designed to have the PEG molecules attached to liposomes to increase their stability in the eye and prolong residence time of the drug. Dextenza, once inserted in the canaliculus, dissolves over a period of about 30 days (for complete dissolution it takes about 90 days), releasing the drug as it dissolves before becoming small enough to fall down the canalicular passage. The process of dissolution is gradual and it starts at the surface of the implant and then continues towards the center.

In some embodiments, the hydrogel implant of the present disclosure is formulated to dissolve and release the at least one therapeutic agent over a period of about 30 days to about 90 days. In some embodiments, the hydrogel implant of the present disclosure is formulated to dissolve and release the at least one therapeutic agent over a period of about 45 days to about 90 days. In some embodiments, the hydrogel implant of the present disclosure is formulated to dissolve and release the at least one therapeutic agent over a period of about 60 days to about 90 days. In some embodiments, the hydrogel implant of the present disclosure is formulated to dissolve and release the at least one therapeutic agent over a period of about 75 days to about 90 days. In some embodiments, the hydrogel implant of the present disclosure is formulated to dissolve and release the at least one therapeutic agent over a period of about 30 days to about 75 days. In some embodiments, the hydrogel implant of the present disclosure is formulated to dissolve and release the at least one therapeutic agent over a period of about 30 days to about 60 days. In some embodiments, the hydrogel implant of the present disclosure is formulated to dissolve and release the at least one therapeutic agent over a period of about 30 days to about 45 days. In some embodiments, the hydrogel implant of the present disclosure is formulated to dissolve and release the at least one therapeutic agent over a period of about 45 days to about 75 days.

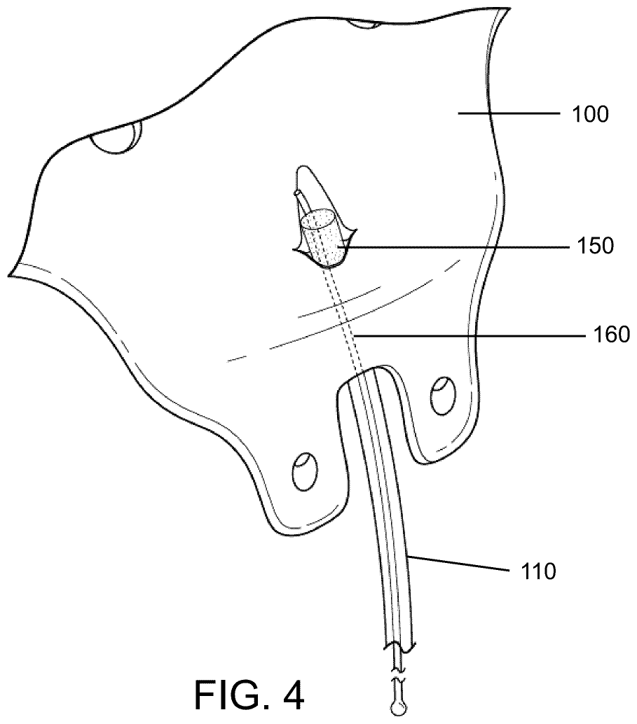

As shown in FIG. 4, embodiments of the present disclosure also include a hydrogel implant 150 formulated to occupy a distal portion of a lumen 110 of a glaucoma drainage device 100, and which includes a ripcord 160. In some embodiments, the implant 150 is in contact with the ripcord 160, such that pulling the ripcord facilitates removal of the implant from the lumen of the glaucoma drainage device. In some embodiments, use of a ripcord provides a mechanism for removing an implant if, for example, it does not dissolve to the level required to allow for fluid to drain from the lumen.

### 3. Kits and Systems for Glaucoma Drainage Devices

In accordance with the above embodiments, the present disclosure also provides a system or kit for the treatment of glaucoma, which comprises a hydrogel implant containing a therapeutic agent(s), and a glaucoma drainage device. In some embodiments, the glaucoma drainage device comprises a drainage tube and a plate for insertion into a subject's eye. In some embodiments, the system or kit further comprises a ripcord to facilitate the removal of the implant from the glaucoma drainage device, for example, in the event the implant does not fully dissolve. As described further herein (FIGS. 1-4), the kits and systems of the present disclosure include a hydrogel implant designed for use with any glaucoma drainage devices (e.g., Ahmed Clearpath 350, or Baerveldt 350). The drainage tube of the glaucoma drainage device has a proximal end that is the entry point for fluid drainage from the eye, as well as a distal end that is positioned near the plate. In some embodiments, the distal end of the drainage tube can be configured to have a modified shape or structure that is expanded as compared to the diameter of the rest of the tube. In this manner, the distal end of the drainage tube is adjustable to accommodate, for example, the type, dosage, or delivery kinetics of the therapeutic agent(s) being delivered to the subject's eye. In some embodiments, the distal end of the drainage tube can be coupled to a reservoir that contains the therapeutic agent(s) in order to deliver an increased amount or concentration of the therapeutic agent(s).

In some embodiments of the kits or systems, the distal end of the drainage tube can include a hydrogel implant. For example, as shown in FIGS. 2 and 3, the hydrogel gel implants of the present disclosure are configured for insertion into the distal end of a drainage tube of a glaucoma drainage device. In some embodiments, the hydrogel comprises polyethylene glycol (PEG). In some embodiments, the PEG comprises polyethylene glycol diacrylate (PEGDA), polyethylene glycol dimethyl acrylate (PEGDMA), and/or polyethylene glycol divinyl ether (PEGDVE), or any derivatives thereof. As would be understood by one of ordinary skill in the art based on the present disclosure, various PEG formulations can be used to generate the hydrogel implants of the present disclosure, and the precise hydrogel PEG formulation depends on the type of glaucoma drainage device, the type of therapeutic agent that is formulated in the implant, the specific dissolving rates desired, and the like.

In accordance with these embodiments, the kits or systems of the present disclosure include hydrogel implants formulated with a therapeutic agent(s) designed to treat one or more systems in a subject's eye, such as a symptom is associated with glaucoma. In some embodiments, the symptom is associated with a disease or disorder that is not characteristic of glaucoma. In other embodiments, the implant can also be formulated with a pharmaceutically acceptable carrier, adjuvant, and/or excipient. In some embodiments, the hydrogel implant is formulated to dissolve over a given period of time (e.g., the length of time for a glaucoma drainage device, after being implanted into a subject's eye, to stabilize in position). This desired length of time for the therapeutic agent to dissolve will be among the factors affecting the particular formulation of the hydrogel. In some embodiments, the hydrogel implant will dissolve and release the therapeutic agent into a subject's eye, and after the implant dissolves, the drainage tube of the glaucoma drainage device provides an unobstructed path for fluid to drain from the device.

As would be understood by one of ordinary skill in the art based on the present disclosure, the therapeutic agent or agents that can be included in the hydrogel implants of the present disclosure can include any therapeutic agent that has been shown to be safe for administration to a subject's eye. For example, in some embodiments, the therapeutic agent included in the implants of the present disclosure is an antibiotic, a small molecule inhibitor, a steroid, and/or a biologic drug (e.g., antibody). In some embodiments, the antibiotic is selected from the group consisting of Tobramycin, Vancomycin, Ciprofloxacin, Ofloxacin, and Moxifloxacin. In some embodiments, the small molecule inhibitor is selected from the group consisting of Rhopressa, Timolol, Brimonidine, and Dorzolamide. In some embodiments, the biologic drug is an antibody selected from bevacizumab and ranibizumab.

### 4. Methods of Generating and Testing Implants

Embodiments of the present disclosure also include a method of generating a glaucoma drainage device comprising a hydrogel implant, which includes positioning the hydrogel implant into a distal portion of a drainage tube of the glaucoma drainage device. In some embodiments, the implant comprises at least one therapeutic agent. One exemplary process for generating a hydrogel according to the methods of the present disclosure is shown in FIG. 5. In accordance with this embodiment, the hydrogel implants of the present disclosure can be incorporated into the distal end of a drainage tube of an Ahmed Clearpath 350 glaucoma drainage device. In one embodiment, Dextenza can be used as hydrogel implant.

In accordance with these embodiments, various percentages of PEG correspond to different swelling ratios, which can inform the choice of a particular hydrogel formulation over another. For example, as shown in FIG. 6, as the percentage of PEG increases (e.g., PEGDA) from 60% to 90%, the percent swelling decreases (over the four timepoints shown). Therefore, PEG percentages can be varied to produce different swelling rates, which can influence the process for generating the hydrogel implants of the present disclosure.

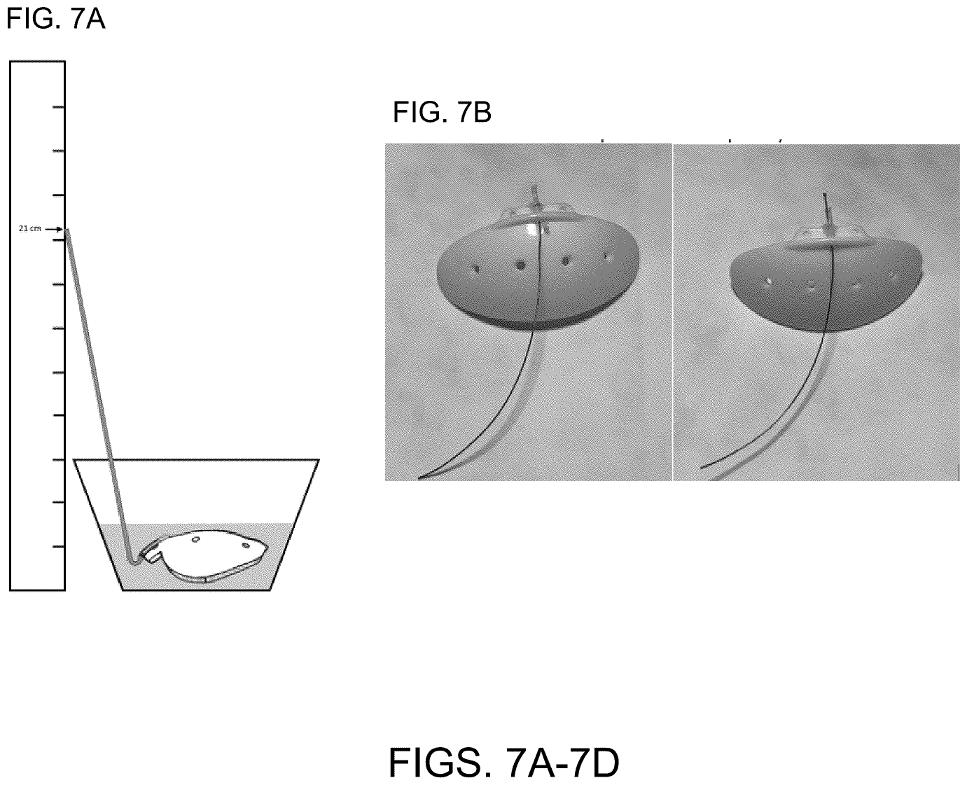

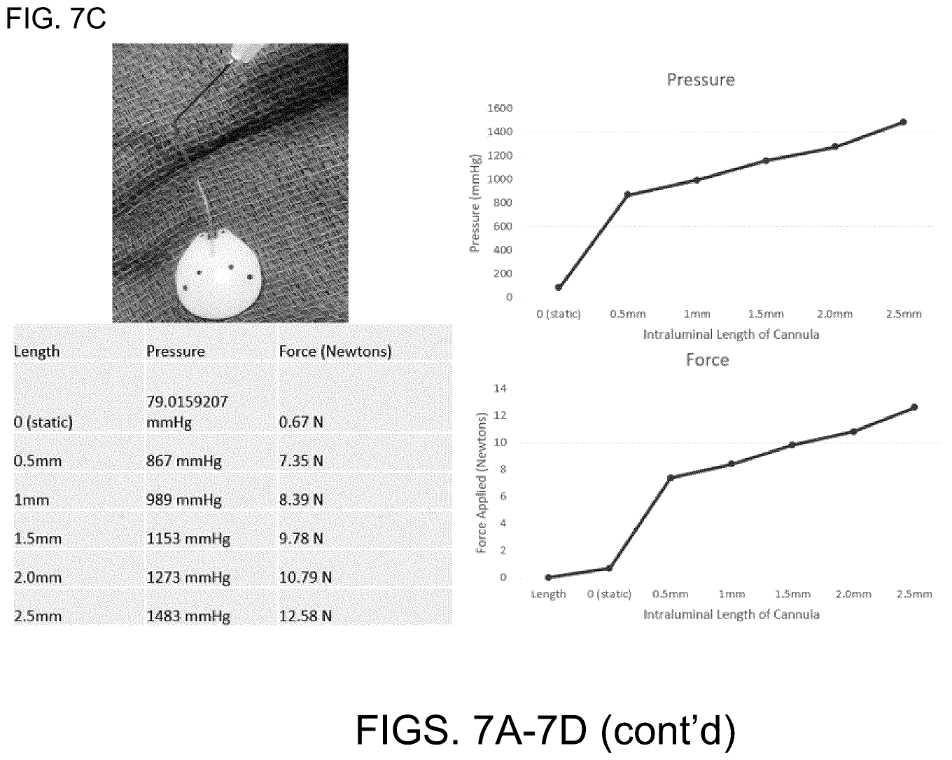

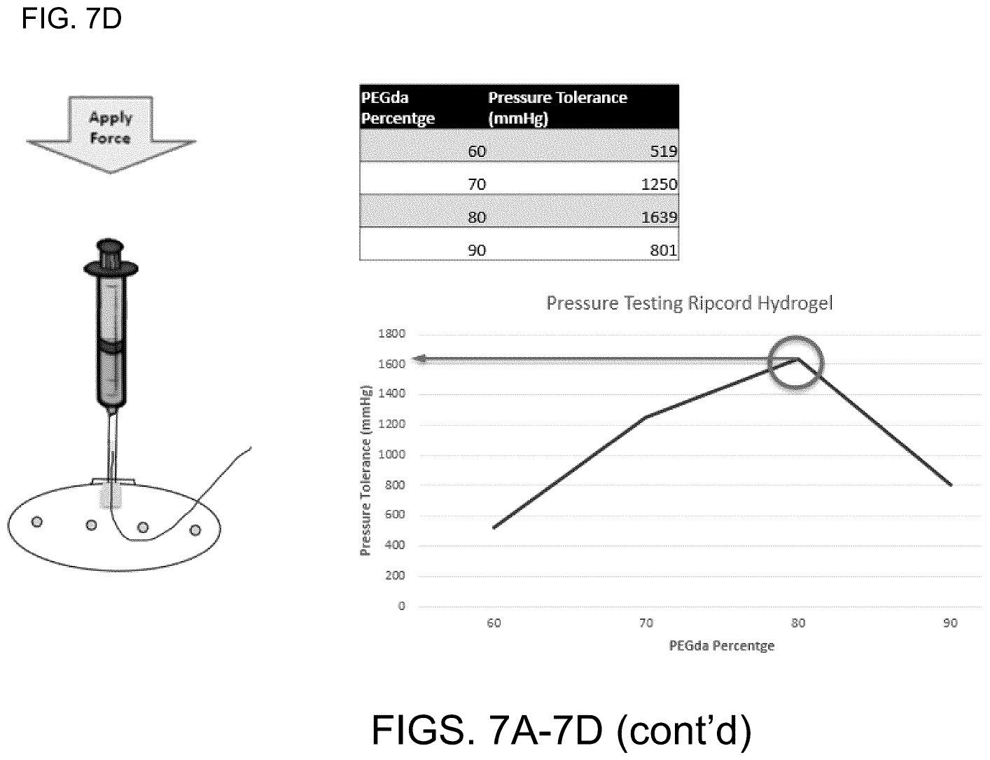

Experiments were conducted to develop a testing device designed to simulate intraocular pressures (IOP) in the subconjunctival space exerted on a glaucoma drainage device (GDD) (FIGS. 7A-7B). A sample of Dextenza was trimmed to fit within the lumen of a glaucoma drainage device. A cannula was attached to a balanced salt solution (BSS) and a filled syringe was then used to prime the tube and to expand the Dextenza implant. The implant was able to withstand strong manual forceful pressures beyond what was previously considered possible. The device was also tested using an Alcon Centurion set to a pressure of 110 mmHg. Both methods were not able to move the implant following its rapid expansion within the drainage tube after priming.

After priming the device, experiments were conducted to test the resiliency of the implant and its eventual dissolution. A pressure of 15 mmHg was used for the simulation as it was reported in the literature to be the average post-operative pressure after non-valved GDD placement. An IOP of 15 mmHg translated to 21 cmH₂O so a BSS-filled tube was attached to the GDD and raised to a height of 21 cmH₂O (FIGS. 7A-7B). The plate was then placed under a shallow dish of water to simulate the subconjunctival space (FIG. 7A). A recording device was then placed and a time-lapse was recorded to show the dissolution of the implant over time.

To verify the robustness of the hydrogel implants of the present disclosure, experiments were conducted to test their ability to withstand intraocular pressures in the subconjunctival space exerted on a glaucoma drainage device (FIGS. 7A-7B). For example, FIG. 7C includes representative results of pressure testing of the hydrogel implant within the lumen of the drainage device, and FIG. 7D includes results of pressure testing with a ripcord in place (FIG. 7D). These results demonstrate that hydrogel implants of the present disclosure (e.g., Dextenza implants) can be formulated to withstand intraocular pressures in the subconjunctival space exerted on a glaucoma drainage device. In some embodiments a hydrogel implant of the present disclosure can be formulated to remain stationary in the lumen of the glaucoma drainage device at pressures up to and including 400 mmHg. In some embodiments a hydrogel implant of the present disclosure can be formulated to remain stationary in the lumen of the glaucoma drainage device at pressures up to and including 300 mmHg. In some embodiments a hydrogel implant of the present disclosure can be formulated to remain stationary in the lumen of the glaucoma drainage device at pressures up to and including 200 mmHg. In some embodiments a hydrogel implant of the present disclosure can be formulated to remain stationary in the lumen of the glaucoma drainage device at pressures up to and including 100 mmHg.

In accordance with the various embodiments described herein, the hydrogel implants of the present disclosure confer various benefits over currently available glaucoma drainage devices. For example, one benefit is being able to reduce drop burden. Particularly in the geriatric and pediatric population, drop burden is crucial to compliance and quality of life. In particular, reducing the need for steroid drops that are often needed for at least a month and needs tapering during the post-operative period, will help both these populations obtain better outcomes and improve quality of life. In both these populations, reducing the frequency of eye drop placement can also reduce the risk of injury to the bleb and surgical site during opening and closing of lids (particularly in children resistant to eye drops). Another benefit is having a localized release of a therapeutic agent (e.g., steroid). Using an implant in the lumen of the tube will help localize the steroid to the site of the surgery and help prevent early fibrosis and scarring at the site of the GDD. Reducing steroid release to the rest of the eye can help prevent elevated pressures from steroid response, which is particularly important for this patient population. Another benefit is reducing surgical operating time as placement of the implant is as simple as threading a rip cord in the lumen of the GDD. A surgeon would then only need to prime the tube and the implant would rapidly expand and seal the tube. There would be no need for tying a dissolvable suture over the tube, risking injury to the tube and increasing surgery time. Reducing the time under surgery provides several benefits to both the patient and the surgeon. Another benefit is that the use of PEG-based hydrogel implants can give rise to the use of many eluting drugs such as ocular hypotensive medications or in combination of steroid medications. The placement of the implant at the lumen helps to reduce endothelial injury which can be seen in other implantable hypotensive medications such as Durysta. Taken together, the use of the lumen space in GDD devices poses a new avenue for implantable and self-dissolving medications that will help improve quality of care for patients, allows for further innovation, and increases efficiency within the operating room.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A hydrogel implant for treating glaucoma in a human subject, wherein the implant comprises at least one therapeutic agent and is formulated to occupy a portion of a lumen of a glaucoma drainage device.

2. The implant of claim 1, wherein the hydrogel comprises polyethylene glycol (PEG).

3. The implant of claim 2, wherein the PEG comprises polyethylene glycol diacrylate (PEGDA), polyethylene glycol dimethyl acrylate (PEGDMA), and/or polyethylene glycol divinyl ether (PEGDVE), or any derivatives thereof.

4. The implant of claim 2, wherein the PEG comprises a molecular weight ranging from 100 to about 20,000.

5. The implant of claim 2, wherein the PEG comprises from about 40% to about 90% PEG.

6. The implant of claim 1, wherein the implant comprises riboflavin-5-phosphate and/or triethanolamine.

7. The implant of claim 1, wherein the at least one therapeutic agent is selected from an antibiotic, a small molecule inhibitor, a steroid, and/or a biologic drug.

8. The implant of claim 7, wherein the antibiotic is selected from the group consisting of Tobramycin, Vancomycin, Ciprofloxacin, Ofloxacin, and Moxifloxacin.

9. The implant of claim 7, wherein the small molecule inhibitor is selected from the group consisting of Rhopressa, Timolol, Brimonidine, and Dorzolamide.

10. The implant of claim 7, wherein the biologic drug is an antibody selected from bevacizumab and ranibizumab.

11. The implant of claim 1, wherein the implant is formulated to remain stationary in the lumen of the glaucoma drainage device at pressures up to and including 400 mmHg.

12. The implant of claim 1, wherein the glaucoma drainage device comprises a plate and a drainage tube, and wherein the implant is formulated to occupy a distal portion of the lumen of the drainage tube.

13. The implant of claim 1, wherein the implant is formulated to dissolve and release the at least one therapeutic agent over a period of about 30 days to about 90 days.

14. The implant of claim 1, wherein the implant is in contact with a ripcord, wherein pulling the ripcord facilitates removal of the implant from the lumen of the glaucoma drainage device.

15. A glaucoma drainage device for treating glaucoma in a human subject comprising a hydrogel implant formulated to occupy a portion of a lumen of a glaucoma drainage device wherein the implant comprises at least one therapeutic agent.

16. The device of claim 15, wherein the glaucoma drainage device comprises a plate and a drainage tube, and wherein the implant is formulated to occupy a distal portion of the lumen of the drainage tube.

17. The device of claim 15, wherein the hydrogel comprises polyethylene glycol diacrylate (PEGDA), polyethylene glycol dimethyl acrylate (PEGDMA), and/or polyethylene glycol divinyl ether (PEGDVE), or any derivatives thereof.

18. The device of claim 15, wherein the at least one therapeutic agent is selected from an antibiotic, a small molecule inhibitor, a steroid, and/or a biologic drug.

19. The device of claim 15, wherein the implant is formulated to remain stationary in the lumen of the glaucoma drainage device at pressures up to and including 400 mmHg, and wherein the implant is formulated to dissolve and release the at least one therapeutic agent over a period of about 30 days to about 90 days.

20. The device of claim 15, wherein the implant is in contact with a ripcord, wherein pulling the ripcord facilitates removal of the implant from the lumen of the glaucoma drainage device.
